(19) United States
(12) Patent Application Publication
(10) Pub. No.: US 2020/0138930 A1
(43) Pub. Date: May 7, 2020

(54) ATTENUATED FISH VACCINE FOLLOWING CULTURE IN IRON LIMITED MEDIA

(71) Applicant: University of Idaho, Moscow, ID (US)

(72) Inventor: Kenneth D. Cain, Moscow, ID (US)

(73) Assignee: University of Idaho, Moscow, ID (US)

(21) Appl. No.: 16/682,892

(22) Filed: Nov. 13, 2019

Related U.S. Application Data
(63) Continuation of application No. 13/570,303, filed on Aug. 9, 2012, now abandoned.
(60) Provisional application No. 61/527,198, filed on Aug. 25, 2011.

(51) Int. Cl. A61K 39/02

(52) U.S. Cl. CPC A61K 39/0216; A61K 2039/522

(57) ABSTRACT

Protection of fish from bacterial disease by vaccination with a live attenuated strain of the causative bacterium is enhanced when the attenuated strain has been grown in an iron-limited medium.

This application is a continuation of U.S. patent application Ser. No. 13/570,303, filed on Aug. 9, 2012, which claims priority from pending U.S. Provisional Patent Application No. 61/527,198 filed on Aug. 25, 2011.

It is hereby acknowledged that the U.S. Government has certain rights in the invention described herein, which was supported in part by United States Department of Agriculture Western Regional Aquaculture Center Budget No. GWK062.

### FIELD OF THE INVENTION

This invention pertains to the field of vaccines to protect fish from disease caused by bacteria. In a particular embodiment, the invention pertains to the field of vaccines to protect fish from disease caused by Flavobacterium psychrophilum.

### BACKGROUND OF THE INVENTION

Flavobacterium psychrophilum is a Gram-negative bacterial fish pathogen that causes bacterial coldwater disease (CWD) and is considered to be one of the most important pathogens affecting salmonid aquaculture due to its wide distribution and economic impact. In the United States, it is estimated that annual losses incurred from CWD in the Pacific Northwest alone are approximately 9.6 and 4 million dollars for commercial aquaculture of rainbow trout (Oncorhynchus mykiss Walbaum) and conservation aquaculture of salmonid species, respectively.

Preventative measures include the use of management strategies to reduce risk factors such as stress, poor water quality, and cutaneous lesions. Even with these in place, CWD commonly occurs and generally requires treatment. Treatment options are limited and include reducing pathogen concentrations, eliminating the spread of the pathogen, and the use of antibiotics. However, the effectiveness of treatment is usually inconsistent, and there are potential risks of developing antibiotic resistant strains. Therefore, a vaccine to prevent CWD is desired. However, even though the need is great and has long been sought, no vaccines for CWD are currently available.

In order to be commercially useful, a vaccine for fish must be capable of conferring protective immunity against a pathogen when the vaccine is administered by practical methods, such as immersing the fish in water containing the vaccine. Vaccination protocols that require individual handling of fish, such as by injection are not practical for most commercial aquaculture operations.

Immunization with killed bacteria has been attempted with F. psychrophilum, and protection obtained by immersion or by injection with the killed bacteria has been minimal. Better protection has been obtained by administering the killed bacteria by injection in combination with an emulsified adjuvant. However, because such vaccination protocols require individual handling of fish, they are less suitable for most aquaculture applications.

Recently, live attenuated bacterial vaccines have been developed to immunize fish against several bacterial diseases. Direct and random approaches can be used to induce mutations into bacterial pathogens to achieve attenuation. Direct approaches include mutation or deletion of genes involved in metabolic pathways and/or pathogenesis, while random approaches include genetic methods such as transposon mutagenesis or the use of chemicals such as antibiotics. In the latter method, bacteria are cultured on or in a medium containing a chemical compound that induces one or more non-lethal mutations in the bacteria, while maintaining the protective immunogenicity of the bacteria.

Attenuated live bacterial vaccines for diseases affecting fish are disclosed in Klesius, U.S. Pat. No. 6,019,981; Shoemaker, U.S. Pat. Nos. 6,881,412; and 6,991,793; Evans, U.S. Pat. No. 7,067,122; and Cain, U.S. Pat. No. 7,740,864.

Klesius discloses an attenuated live bacterial vaccine against enteric septicemia of catfish caused by Edwardsiella ictaluri. Shoemaker discloses an attenuated live bacterial vaccine against Flavobacterium columnare, the causative agent of columnaris disease. Evans discloses an attenuated live bacterial vaccine against Edwardsiella tarda, the causative agent of Edwardsiella septicemia disease. Cain discloses an attenuated live bacterial vaccine against coldwater disease caused by Flavobacterium psychrophilum. Each of these attenuated live vaccines of Klesius, Shoemaker, and Evans has been shown to be effective when administered to fish by immersion, therefore obviating the need for individual handling of fish.

### DESCRIPTION OF THE DRAWINGS

FIG. 1 is a graph comparing the percent survival of injection-vaccinated fish following challenge with a virulent strain of Flavobacterium psychrophilum. In FIG. 1, ∘ are data points for mock vaccinated fish injected with vehicle, ▾ are data points for fish vaccinated by injection with a live attenuated strain F. psychrophilum that had been grown in an iron-limited medium, and ♦ are data points for fish vaccinated by injection with the live attenuated strain of F. psychrophilum that had been grown in a growth medium that was not iron-limited.

FIG. 2 is a graph comparing the percent survival of immersion-vaccinated fish following challenge with a virulent strain of Flavobacterium psychrophilum. In FIG. 1, ● are data points for mock vaccinated fish immersed with vehicle, ▪ are data points for fish vaccinated by immersion with a live attenuated strain F. psychrophilum that had been grown in an iron-limited medium, and ▴ are data points for fish vaccinated by immersion with the live attenuated strain of F. psychrophilum that had been grown in a growth medium that was not iron-limited.

### DESCRIPTION OF THE INVENTION

It has been discovered that protection of fish from bacterial disease by vaccination with a live attenuated strain of the causative bacterium is enhanced when the attenuated strain has been grown in an iron-limited medium.

The term “iron-limited” when referring to a bacterial growth medium means a medium that is essentially free of iron or, if iron is present in the medium, contains an iron chelator at a concentration that if sufficient to reduce the amount of free iron in the medium.

In one embodiment, the invention is a method for producing a vaccine for protecting fish against a disease caused by a bacterium. According to this embodiment of the invention, a live attenuated strain of the bacterium is grown in or on an iron-limited medium and the bacterium is then harvested. In a preferred embodiment, when the attenuated strain of the bacterium has been grown on or in a medium that is not iron-limited is effective when used as a vaccine in reducing morbidity and/or mortality due to disease caused by exposure of the fish to a live non-attenuated pathogenic strain of the bacterium. Growing the attenuated strain of the bacterium in or on an iron-limited medium provides an increased efficacy of the vaccine in reducing morbidity and/or mortality compared to the efficacy of a similar vaccine made of the same strain of bacterium but grown in or on a medium that is not iron-limited.

In one embodiment, the invention is a vaccine for protecting fish against a disease caused by a bacterium where the vaccine comprises a live attenuated strain of the bacterium which has been grown in or on an iron-limited medium, wherein the live attenuated strain of the bacterium which has been grown on or in a medium that is not iron-limited is effective in reducing morbidity and/or mortality due to disease caused by exposure of the fish to a live non-attenuated pathogenic strain of the bacterium. The vaccine comprising the live attenuated strain of the bacterium that has been grown in or on an iron-limited medium is more effective than a similar vaccine comprising the live attenuated strain of the bacterium that has been grown in or on a medium that is not iron-limited.

In another embodiment, the invention is a method for immunizing fish against a bacterial disease. According to this embodiment of the invention, fish are vaccinated with an attenuated live bacteria that has been grown in an iron-limited medium, wherein vaccination of the live attenuated strain of the bacterium which has been grown on or in a medium that is not iron-limited is effective in reducing morbidity and/or mortality due to disease caused by exposure of the fish to a live non-attenuated pathogenic strain of the bacterium.

The enhanced protection obtained by vaccination with a bacterial strain that has been grown in or on an iron-limited medium is surprising because iron is an essential element for bacterial growth and, therefore, media on which bacteria are grown generally include iron-containing ingredients. The reason for the enhanced protection is not known, but because iron is typically bound to proteins within host fish and, therefore, is not present in significant amounts in a free form within the host, a growth medium in which free iron is limited is more reflective of the natural host environment than a growth medium in which free iron is abundant. Thus, because a growth medium in which iron is limited or bound may present a more natural environment for a bacterial pathogen than an iron-rich medium and, therefore, increased amounts or different antigens may be expressed by the bacteria, whether pathogenic or attenuated, than would occur when the bacteria are grown in iron-rich media. It is theorized that the antigens produced by bacteria in an iron-poor medium may more closely relate to those produced by a pathogenic bacteria in its natural environment in the host. In this way, limiting the amount of free iron in a growth medium for an attenuated strain results in the production of antigens more closely related to those of the natural infective strain, and thereby produces a higher level of protection against the infective strain.

The free iron in a bacterial growth medium may be limited in several ways. For example, the medium may be constituted solely or essentially of ingredients that are free of iron. Typically, however, bacterial growth media, especially complex media such as those that contain an extract from beef or yeast, contain significant amounts of free iron. When utilizing such a growth medium, free iron may be reduced or substantially eliminated by combining an iron chelator in the medium. Examples of iron chelators that may be included in a bacterial growth medium include 2,2-dipyridyl (DPD, defoxamine (DFO), phytic acid, diethylenetriaminepentacetic acid (DTPA), deferoxamine mesylate (DM), ethylenediaminetetraacetic acid (EDTA), and ethylenediamine-N,N′-diacetic acid (EDDA).

In order to test this conception, an attenuated strain of a bacterium known to cause disease in fish was grown (a) in an iron-limited medium or (b) in an iron-rich medium, and fish were then vaccinated with the strain grown in one of these two media. The levels of protection due to the two vaccination protocols were compared. It was determined that vaccination with the attenuated bacteria grown either in iron-limited or iron-rich media protected the fish against disease compared to negative control fish that were vaccinated with vehicle. However, protection, determined by percentage survival following challenge, was greater in fish vaccinated with the attenuated strain grown in iron-limited media compared to those vaccinated with the same attenuated strain grown in iron-rich media.

The pathogenic bacteria that may be vaccinated against according to the present application include any bacterium that is known to cause disease in fish and for which a degree of protection against disease caused by that bacterium may be induced by vaccination of a susceptible animal host with an attenuated strain of the bacterium. For example, protection conferred by vaccination with attenuated strains that are effective in protecting against diseases such as enteric septicemia of catfish, columnaris disease, Edwardsiella septicemia disease, or coldwater disease may be improved by growing the attenuated strains in an iron-limited medium.

In the present application, coldwater disease, a disease that causes significant mortality in salmonids, is utilized as an example. However, one of skill in the art will understand that the concept of growing an attenuated strain of a pathogenic bacterium, which attenuated strain is capable of reducing the morbidity or mortality due to the pathogenic bacterium, in a medium that has limited iron availability, is applicable to any pathogenic bacterium that may be protected against by vaccination with an attenuated strain.

The medium in or on which the attenuated strain is grown is any medium that will support growth of the bacterial strain. For example, tryptone yeast extract salt (TYES) in liquid or solid form may be utilized. Other examples include tryptic soy liquid or solid medium (TSB or TSA), brain heart infusion agar or broth, Cytophaga agar or broth, Anacker and Ordal liquid or solid medium, Hsu-Shotts liquid or solid medium, MAT liquid or solid medium, and Shieh liquid or solid medium.

The growth medium may be substantially free of iron, or the medium may contain iron and further contain an iron-chelating agent in an amount or concentration that is sufficient to reduce the amount of free iron in the medium. Examples of suitable iron-chelating agents include 2,2-dipyridyl (DPD, defoxamine (DFO), phytic acid, diethylenetriaminepentacetic acid (DTPA), deferoxamine mesylate (DM), ethylenediaminetetraacetic acid (EDTA), and ethylenediamine-N,N′-diacetic acid (EDDA).

The attenuated bacteria may be, or may have been, attenuated by any method by which the virulence of the bacteria may be reduced or eliminated. For example, the bacteria may be attenuated by exposing a wild-type strain of the bacteria to radiation or to a chemical compound that promotes mutations. Antibiotics, such as rifampicin may be used for the development of attenuated strains of infectious bacteria. Such methods are well known in the art.

The bacteria of the present application are “attenuated” which term is used herein to mean that the bacteria has reduced virulence compared to that of wild-type non-attenuated bacteria of the same species. Preferably, the attenuated bacteria have no capability to cause disease and such bacteria may be referred to “completely attenuated.”

Attenuated bacteria that are suitable for the method of this application include any attenuated bacteria that are effective to provide protection against disease caused by non-attenuated bacteria of the same species when administered to fish. In a preferred illustrative embodiment, the attenuated bacteria are Flavobacterium psychrophilum. Other examples of bacteria suitable for the method of the present application include attenuated strains of Edwardsiella ictaluri, Flavobacterium columnare, and Edwardsiella tarda.

The method of the present application may be used to protect any fish that is susceptible to infection and disease caused by a particular bacterial organism. The fish may be a marine or salt-water fish. Examples of suitable fish for the method of invention include salmonids (Oncorhynchus sp. and Salmo sp.), American, European, and Japanese eels (Anguilla sp.), tilapia (Oreochromis sp.), striped bass and hybrid-striped bass (Morone chrysops. and M. saxatilis), flounders (Seriola sp.), seabream (Sparus sp.), sea perch (Lates calcarifer), the estuarine grouper (Epinephelus tawine), walleye (Stitzostedion vitreum), channel catfish (Ictalurus punctutus), centrachids (such as largemouth bass, Micropterus salmoides), brown bullheads (Nebulosus sp.), fat head minnows (Pimephales promelas), golden shiners (Netemigonus crysoleucas), goldfish (Carassius auratus), carp (Cyprinus carpio), and aquarium fish species such as black mollies (Poecilia sphenops) and platies (Xiphophorus maculatus). Species affected specifically by CWD include all salmonids. The pathogen has also been reported in non-salmonid species, such as eel Anguilla sp., sea lamprey Petromyzon marinus, carp Cyprinus carpio, tench Tinca tinca, crucian carp Carassius carassius, goldfish C. auratus, ayu Plecoglossus altivelis, pale chub Zacco platypus, perch Perca fluviatilis, and roach Rutilus rutilus.

The attenuated vaccine is administered to the fish in any manner in which the vaccine is effective. For example, the vaccine may be administered by injection, such as intraperitoneal injection. Preferably, the attenuated vaccine is administered by mass vaccination, which refers to methods of vaccination that do not require handling of individual fish. Methods of mass vaccination include oral, spray, and immersion delivery.

The invention is disclosed further in the following non-limiting examples. In the examples that follow, coldwater disease and Flavobacterium psychrophilum are utilized as illustrative examples of a disease and a bacterium that are suitable for the invention. One skilled in the art will understand that this is merely an illustration and that the invention is applicable to other diseases of fish and other pathogenic bacteria that cause disease in fish, and particularly to diseases of fish in which an attenuated bacterial vaccine may be used to protect fish from disease caused by a non-attenuated strain of the bacteria.

### Example 1—Culture Methods

Flavobacterium psychrophilum strains were cultured for 72-96 h at 15° C. in 250 ml tryptone yeast extract salts broth (TYES; 0.4% tryptone, 0.04% yeast extract, 0.05% calcium chloride, 0.05% magnesium sulfate, pH 7.2) for vaccinations and challenges. To prepare iron-limited media (ILM), the iron chelator, 2′2-dipyridyl (DPD) (Sigma-Aldrich, St. Louis, Mo., USA), was added to TYES broth at a final concentration of 50 M. For the challenge trial, a virulent strain of F. psychrophilum, CSF-259-93, was grown statically. For the attenuated strain, F. psychrophilum strain 259-93.B.17 (ATCC Accession No. PTA-9205) was grown on an orbital shaker at ˜83 rpm. To harvest bacteria for both the injection immunization and challenge, cultures were centrifuged at 4300×g for 15 min, the supernatant was poured off, and pellets were re-suspended in sterile phosphate buffered saline (PBS). To estimate CFU ml⁻¹, a 6×6 drop plate method using TYES agar plates was used. Plates were incubated at 15° C. for 96 h and colonies were counted.

### Example 2—Vaccine Trials

### Example 2.a.—Injection Vaccination

A total of 540 Coho salmon (Oncorhynchus kisutch) were divided into three groups of 180 fish each (mean weight 3.6 g) and placed into two treatment groups and one control group. The fish were anesthetized in 100 μg ml⁻¹ tricaine methanesulfonate (MS-222, Argent Chemicals, Redmond, Wash., USA) and intraperitoneally (i.p.) injected using a 30 gauge needle. The control group received 50 μl of sterile PBS as a mock immunization administered by peritoneal injection. Fish in the prior art vaccination group were injected intraperionteally with 50 μl of 1.0×10⁷ CFU fish⁻¹ of strain CSF259-93.B17, a strain that is effective in protecting fish against coldwater disease, as disclosed in Cain, U.S. Pat. No. 7,740,864. Fish vaccinated according to the method of the present application were injected intraperitoneally with 50 μl of 3.3×10⁴ CFU fish⁻¹ of strain 259-93.B.17 grown in iron-limited medium, which strain is referred to as “259-93.B.17 ILM”. Fish were booster immunized at 4 weeks post-immunization. As before, the control group received 50 μl PBS while the two treatment groups were i.p. injected with 50 μl resuspended bacteria at optical densities corresponding to 1.0×10⁶ CFU fish⁻¹ (259-93.B.17) and 8.9×10⁴ CFU fish⁻¹ (259-93.B.17 ILM).

### Example 2.b.—Immersion Vaccination

A total of 540 Coho salmon (Oncorhynchus kisutch) were divided into three groups of 180 fish each (mean weight 3.6 g) and placed into two treatment groups and one control group. The fish were anesthetized in 100 μg ml⁻¹ tricaine methanesulfonate (MS-222, Argent Chemicals, Redmond, Wash., USA) prior to removal of the adipose fin and briefly allowed to recover before immunization. All groups were immersed for 1 h with aeration. The control treatment was sterile TYES broth diluted 1:4 with tank water. Fish in the 259-93.B.17 treatment were immersed in water containing a final concentration of 5.2×10⁷ CFU ml⁻¹. Fish in the 259-93.B.17 ILM treatment were immersed in a solution of water containing 5.1×10⁶ CFU ml⁻¹. Fish were booster immunized at 4 weeks post-immunization by immersion in the appropriate treatment for 1 hr with aeration. Booster doses were 5.1×10⁶ CFU ml⁻¹ (259-93.B.17) and 9.8×10⁴ CFU ml⁻¹ (259-93.B.17 ILM). The control group was mock booster immunized as described above.

### Example 3—Bacterial Challenge

At six weeks post-immunization, triplicate groups of 25 fish from the two vaccinated groups from both the injection vaccinated fish and the immersion vaccinated fish and from the mock vaccinated groups of Examples 1 and 2 were subcutaneously injected with 25 μl of the parent strain F. psychrophilum CSF259-93 at a concentration of approximately 7.1×10⁵ CFU fish⁻¹. In each group, a subset of fish (n=20) were injected with sterile PBS to serve as the mock infected control. Mortalities were monitored on a daily basis for 28 days.

### Example 4—Results

### Example 4.a.—Injection Vaccination

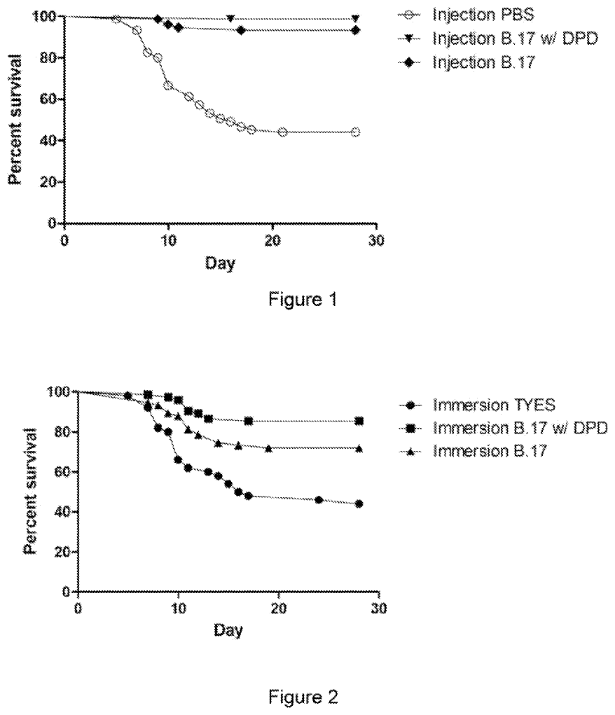

For the injection delivery trial of Example 2.a, the results of immunization with either the attenuated strain or the attenuated strain grown in iron-limited medium are shown in FIG. 1 and in Table 1. FIG. 1 shows the data in terms of percent survival up to 30 days post challenge and Table 1 shows the data in terms of cumulative percent mortality (CPM), which is the reciprocal of percent survival, at six weeks post challenge.

TABLE 1

| Vaccination Treatment | Cumulative | Relative |
|---|---|---|
| (Immersion) | Percent Mortality | Percent Survival |
| Mock Immunized | 65.3% | |
| 259-93.B.17 | 6.7% | 90% |
| 259-93.B.17 ILM | 1.3% | 98% |

As shown in Table 1, vaccination was effective in reducing mortality due to challenge with a virulent bacterial strain compared to control when the strain that was utilized was either the attenuated strain grown in a non-iron-limited growth medium (259-93.B. 17) or the attenuated strain grown in an iron-limited growth medium (259-93.B. 17 ILM). Percent mortality was reduced from 65.3% to 6.7% by vaccination with the attenuated strain grown in a non-iron-limited medium, which is calculated to be a 90% relative survival rate with vaccination compared to negative control. However, protection conferred by vaccination with the same attenuated strain but grown in an iron-limited medium was even more effective. The percent mortality was reduced from 65.3% to only 1.3%, a 98% relative survival rate compared to negative control.

Compared to vaccination with the attenuated strain grown in a medium in which iron was not limited, vaccination with the attenuated strain grown in iron-limited medium reduced mortality following challenge from 6.7% to only 1.3%, which is calculated to be an 80% increase in relative survival rate between these two groups.

Similar data is shown in FIG. 1, which shows higher survival following vaccination with an attenuated bacterial strain grown in either iron-rich or iron-limited media compared to control and that survival was highest in fish vaccinated with the attenuated strain grown in iron-limited medium.

### Example 4.b.—Immersion Vaccination

For the immersion delivery trial of Example 2.b, the results of immunization with either the attenuated strain or the attenuated strain grown in iron-limited medium are shown in FIG. 2 and in Table 2. FIG. 2 shows the data in terms of percent survival up to 28 days post challenge and Table 2 shows the data in terms of cumulative percent mortality (CPM), which is the reciprocal of percent survival, at four weeks post challenge.

TABLE 2

| Vaccination Treatment | Cumulative | Relative |
|---|---|---|
| (Immersion) | Percent Mortality | Percent Survival |
| Mock Immunized | 54 | |
| 259-93.B.17 | 29.3% | 46% |
| 259-93.B.17 ILM | 14.7% | 73% |

As shown in Table 2, vaccination by immersion was effective in reducing mortality due to challenge with a virulent bacterial strain compared to control when the strain that was utilized was either the attenuated strain grown in a non-iron-limited growth medium (259-93.B.17) or the attenuated strain grown in an iron-limited growth medium (259-93.B.17 ILM). Percent mortality was reduced from 54% to 29.3% by vaccination with the attenuated strain grown in a non-iron-limited medium, which is calculated to be a 46% relative survival rate with vaccination compared to negative control. However, protection conferred by vaccination with the same attenuated strain but grown in an iron-limited medium was even more effective. The percent mortality was reduced from 54% to only 14.7%, a 73% relative survival rate compared to negative control.

Compared to vaccination with the attenuated strain grown in a medium in which iron was not limited, vaccination with the attenuated strain grown in iron-limited medium reduced mortality following challenge from 29.3% to only 14.7%, which is calculated to be a 49% increase in relative survival rate between these two groups.

Similar data is shown in FIG. 2, which shows higher survival following vaccination with an attenuated bacterial strain grown in either iron-rich or iron-limited media compared to control and that survival was highest in fish vaccinated with the attenuated strain grown in iron-limited medium.

The invention disclosed in this specification has been illustrated with specific examples, such as particular organism and disease (Flavobacterium psychrophilum and coldwater disease). One skilled in the art will understand that these examples are merely illustrative and that the scope of the invention is as disclosed herein. Additionally, further modifications, uses, and applications of the invention described herein will be apparent to those skilled in the art. It is intended that such modifications be encompassed in the above description and in the following claims.

1-9. (canceled)

10. A method, comprising:
providing a fish vaccine comprising a live attenuated derivative strain of a bacterium grown in or on a medium that is iron-limited from a live attenuated primary strain of the bacterium which had been grown in or on a medium that is not iron-limited; and
administering the fish vaccine to fish to reduce morbidity and/or mortality due to disease caused by exposure of the fish to a live non-attenuated pathogenic strain of the bacterium.

11. The method of claim 10 wherein the disease is coldwater disease caused by Flavobacterium psychrophilum.

12. The method of claim 11 wherein the live attenuated primary strain is Flavobacterium psychrophilum 259-93.B. 17 deposited with the ATCC under the accession number PTA 9205.

13. The method of claim 10 wherein the disease is selected from the group consisting of enteric septicemia of catfish caused by Edwardsiella ictaluri, columnaris disease caused by Flavobacterium columnare, Edwardsiella septicemia disease caused by Edwardsiella tarda, and coldwater disease caused by Flavobacterium psychrophilum.

14. The method of claim 10, comprising:
providing a vaccine comprising a live attenuated derivative strain of Flavobacterium psychrophilum grown in or on an iron-limited medium from a primary live attenuated strain of Flavobacterium psychrophilum which has been grown on or in a medium that is not iron-limited; and
administering the vaccine to fish to reduce morbidity and/or mortality due to coldwater disease caused by exposure of the fish to a live non-attenuated pathogenic strain of Flavobacterium psychrophilum.

15. The method of claim 10, wherein administering the vaccine comprises administering the vaccine at a concentration of at least 3×10⁴ CFU fish⁻¹ of the live attenuated derivative strain of the bacterium.

16. The method of claim 10, comprising administering the vaccine to fish by immersion in water having a concentration of at least 5×10⁶ CFU ml⁻¹ of the live attenuated derivative strain of the bacterium.

17. The method of claim 12, comprising administering the vaccine to fish intraperitoneally at a concentration of at least 3×10⁴ CFU fish⁻¹ of the live attenuated derivative strain of the bacterium.

18. The method of claim 12, comprising administering the vaccine to fish by immersion in water having a concentration of at least 5×10⁶ CFU ml⁻¹ of the live attenuated derivative strain of the bacterium.

19. The method of claim 12, further comprising administering the vaccine in an amount effective to achieve a relative percent survival of greater than 49% in the fish compared to fish that are not administered the live attenuated derivative strain grown in or on iron-limited media.

20. The method of claim 10, wherein administering the fish vaccine comprises administering a first dose of the fish vaccine to a first group of fish, and administering a second dose of the fish vaccine to at least some members of the first group of fish at a time subsequent to the first administration.

21. The method of claim 20, comprising administering the second dose of the fish vaccine 4 weeks after the first dose is administered.

22. A method for reducing morbidity and/or mortality of salmonids due to coldwater disease caused by Flavobacterium psychrophilum, comprising:
providing a fish vaccine comprising a live, attenuated strain of a Flavobacterium psychrophilum designated 259-93.B. 17, deposited with ATCC under the accession number PTA 9205, that is grown in or on iron-limited media; and
administering the fish vaccine to salmonids to reduce mortality and/or morbidity in the salmonids due to coldwater disease caused by a live, non-attenuated pathogenic strain of Flavobacterium psychrophilum relative to mortality and/or morbidity in salmonid fish that have not been administered the fish vaccine and are exposed to the live, non-attenuated pathogenic strain of Flavobacterium psychrophilum.

23. A method, comprising immersing salmonids in water that contains an effective amount of a live, attenuated strain of Flavobacterium psychrophilum grown in or on iron-limited media, thereby increasing a survival rate of the salmonids relative to a survival rate of salmonids that are not immersed in the water and are exposed to a live strain of a bacterium that is not grown in or on iron-limited media.

24. The method of claim 23, wherein the live strain of the bacterium that is not grown in or on iron-limited media is a live strain of Flavobacterium psychrophilum that is not grown in or on iron-limited media.

25. The method of claim 24, wherein the live strain of Flavobacterium psychrophilum that is not grown in or on iron-limited media is a live, non-attenuated and pathogenic strain of Flavobacterium psychrophilum.

26. The method of claim 23, comprising immersing salmonids in water that contains an effective amount of a live, attenuated strain of Flavobacterium psychrophilum designated 259-93.B. 17 deposited with ATCC under the accession number PTA 9205 and grown in or on iron-limited media, thereby increasing a survival rate of the salmonids relative to salmonids that are exposed to a live, non-attenuated pathogenic strain of Flavobacterium psychrophilum and are not immersed in the water containing the live, attenuated strain of Flavobacterium psychrophilum grown in or on iron-limited media.
